(19) United States
(12) Patent Application Publication
(10) Pub. No.: US 2006/0147456 A1
(43) Pub. Date: Jul. 6, 2006

(54) Induction of apoptosis in toll-like receptor expressing tumor cells

(76) Inventors: Serge Lebecque, Civrieux d'Azergues (FR); Toufic Renno, Civrieux d'Azergues (FR); Bruno Salaun, Prilly (CH); Isabelle Coste-Invernizzi, Chazay d'Azergues (FR); Marie-Clotilde Rissoan, Lyon (FR)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/184,191

(22) Filed: Jul. 19, 2005

Related U.S. Application Data
(60) Provisional application No. 60/589,616, filed on Jul. 20, 2004.

(51) Int. Cl. A61K 48/00; A61K 39/395

(52) U.S. Cl. 424/155.1; 514/44

(57) ABSTRACT

Some types of cancer cells express one or more Toll-like receptors (TLRs). These TLRs are therapeutic targets. The invention relates to methods for treating Toll-like receptor expressing cancers and tumor cells by selecting a TLR expressing tumor cell and contacting the cell with a therapeutically effective amount of a TLR ligand. The invention particularly relates to methods for treating TLR3 expressing cancers and tumor cells using TLR3 agonists.

### CROSS-REFERENCE TO RELATED APPLICATIONS

This application claims priority under 35 U.S.C. § 119(e) to U.S. Application Ser. No. 60/589,616 filed Jul. 20, 2004.

### BACKGROUND OF THE INVENTION

1. Field of the Invention

The invention relates to methods for treating Toll-like receptor (TLR) expressing cancers and tumor cells by selecting a TLR expressing tumor cell and contacting the cell with a therapeutically effective amount of a TLR ligand. The invention particularly relates to methods for treating TLR3 expressing cancers and tumor cells using TLR3 agonists.

2. Background

Cancer is one of the leading causes of death in the world. Therefore, it is essential that we develop new methods to treat this deadly disease. Many current cancer therapies affect rapidly dividing cells. These therapies have devastating side effects because they affect all rapidly dividing cells, such as cells of the gastrointestinal tract and hair follicles, and not just cancer cells. Therefore, new methods of treatment are needed that do not have such devastating side effects. The present application identifies Toll-like receptor 3 as a therapeutic target in the treatment of cancer.

Drosophila toll proteins control dorsal-ventral patterning in Drosophila embryos and are also thought to represent an ancient host defense mechanism.

Human homologues of Drosophila toll, called Toll-like receptors (TLRs), have also been identified. Alignment of the sequences of the human and Drosophila Toll proteins shows that there is homology over the entire length of the protein chains. Accordingly, TLRs are believed to be an important component of innate immunity in humans.

The family of human Toll-like receptors is comprised of ten highly conserved receptor proteins, TLR1-TLR10. Like Drosophila toll, human TLRs are type I transmembrane proteins with an extracellular domain consisting of a leucine-rich repeat (LRR) domain that recognizes pathogen-associated molecular patterns (PAMPs), and a cytoplasmic domain that is homologous to the cytoplasmic domain of the human interleukin-1 (IL-1) receptor. Similar to the signaling pathways for both Drosophila toll and the IL-1 receptor, human Toll-like receptors signal through the NF-κB pathway.

Although mammalian TLRs share many characteristics and signal transduction mechanisms, their biologic functions are very different. This is due in part to the fact that four different adaptor molecules (MyD88, TIRAP, TRIF and TRAF) are associated in various combinations with the TLRs and mediate different signaling pathways. In addition, different ligands for one TLR may preferentially activate different signal transduction pathways. Furthermore, the TLRs are differentially expressed in various hematopoietic and non-hematopoietic cells. Accordingly, the response to a TLR ligand depends not only on the signal pathway activated by the TLR, but also on the nature of the cells in which the individual TLR is expressed.

Although ligands for some TLRs remain to be identified, a number of TLR specific ligands have already been reported. For example, Poly IC and Poly AU are both TLR3 agonists.

Polyinosinic-polycytidylic acid (Poly IC) is a high molecular weight synthetic double stranded RNA that is heterogeneous in size. Poly IC is a TLR3 agonist, but is also a potent activator of PKR, a ubiquitous enzyme involved in anti-viral responses and gene post-transcriptional regulation.

Polyadenylic-polyuridylic acid (Poly AU) is a double stranded complex of synthetic polyribonucleotides. Poly AU is a TLR3 agonist. Poly AU is a modulator of both humoral and cellular immune responses, and is also an inducer of interferon.

Although both Poly IC and Poly AU were used in several clinical trials as adjuvant therapy in different types of cancer, such as cancer of the breast, bladder, kidney and stomach, these agents have not been used previously in the novel methods disclosed herein.

As stated previously, the present application identifies Toll-like receptor 3 as a therapeutic target in the treatment of cancer. The following published studies relate to the relationship between TLRs and apoptosis.

Aliprantis et al. reports on experiments examining the effect of bacterial lipoproteins (BLPs) on the induction of apoptosis in a monocytic cell line that expresses human Toll-like Receptor 2 (hTLR2). See Aliprantis et al., “Cell Activation and Apoptosis by Bacterial Lipoproteins Through Toll-like Receptor-2”, Science, vol. 285, pp. 736-739 (Jul. 30, 1999).

Another reference by Aliprantis et al. relates to the role of TLR2 in triggering the activation of caspase 8 through the recruitment of FADD. See Aliprantis et al., “The apoptotic signaling pathway activated by Toll-like receptor-2”, Embo J., vol. 19(13), pp. 3325-3336 (2000).

Sabroe et al. relates to the role of TLR2 in neutrophil survival. See Sabroe et al., “Selective Roles for Toll-Like Receptor (TLR)2 and TLR4 in the Regulation of Neutrophil Activation and Life Span”, J. Immunology, vol.170, pp. 5268-5275 (2003).

Bannerman and Goldblum relate to studies indicating TLR4 and TLR2 as bacterial lipopolysaccharide (LPS) receptors. See Bannerman and Goldblum, “Mechanisms of bacterial lipopolysaccharide-induced endothelial apoptosis”, Am. J. Physiology Lung Cell Molecular Physiology, vol. 284, pp. L899-L914 (2003).

Meyer et al. relates to studies on the induction of apoptosis by a TLR7 agonist in human epithelial cell lines (HeLa S3), keratinocytes (HaCaT and A431 cells) and mouse fibroblasts (McCoy cells). See Meyer et al., “Induction of apoptosis by Toll-like Receptor-7 agonist in tissue cultures”, British J. Dermatology, vol. 149 (supp. 66), pp. 9-13 (2003).

Wen et al. suggest that diabetes is induced, in part, by the combination of direct recognition of the virus-like stimulus by pancreatic islets through the expression of the innate immune receptor, TLR3. Wen et al. also speculate that the induction of apoptosis by Poly IC is possibly mediated by TLR3. See Wen et al., “The Effect of Innate Immunity on Autoimmune Diabetes and the Expression of Toll-Like Receptors on Pancreatic Islets”, J. Immunology, vol.172, pp. 3173-3180 (2004).

Finally, Han et al. relates to the induction of apoptosis in 293 cells overexpressing TRIF. Han et al. also refer to a proposed model for TRIF-induced intracellular signaling pathways (ISRE/IFNβ, NF-κB and apoptosis) that is activated by TLR3. See Han et al., “Mechanisms of the TRIF-induced Interferon-stimulated Response Element and NF-κB Activation and Apoptosis Pathways”, J. Biological Chemistry, vol. 279, no.15, pp. 15652-15661 (2004).

### SUMMARY OF THE INVENTION

An embodiment of the invention provides a method for treating cancer comprising: a) selecting a patient that has a TLR expressing cancer, and b) administering to the patient a therapeutically effective amount of a TLR ligand. Preferably, the ligand is an agonist or an antagonist.

An alternative embodiment of the invention provides a method for inducing apoptosis of a tumor cell comprising: a) selecting a TLR expressing tumor cell, and b) contacting the cell with a TLR ligand in an amount effective to induce apoptosis in the cell. Preferably, the ligand is an agonist or an antagonist.

Another embodiment of the invention provides a method for treating cancer comprising: a) selecting a patient that has a TLR3 expressing cancer; and b) administering to the patient a therapeutically effective amount of a TLR3 ligand. Preferably, the ligand is an agonist or an antagonist. More preferably, the agonist is Poly AU. Most preferably, the agonist is Poly IC. Alternatively, the antagonist is an antibody or fragment thereof. Preferably, the TLR3 expressing cancer is colon cancer. Most preferably, the TLR3 expressing cancer is breast cancer. The method may further comprise administering to the patient a chemotherapeutic agent or a cancer treatment. The method may also further comprise administering to the patient a low dose of type I IFN prior to administration of TLR3 ligand.

An alternative embodiment of the invention provides a method for inducing apoptosis of a tumor cell comprising: a) selecting a TLR3 expressing tumor cell, and b) contacting the cell with a TLR3 ligand in an amount effective to induce apoptosis in the cell. Preferably, the ligand is an agonist or an antagonist. More preferably, the agonist is Poly AU. Most preferably, the agonist is Poly IC. Alternatively, the antagonist is an antibody or fragment thereof. Preferably, the TLR3 expressing tumor cell is a colon cancer cell. Most preferably, the TLR3 expressing tumor cell is a breast cancer cell. The method may further comprise contacting the cell with a chemotherapeutic agent or a cancer treatment. The method may also further comprise contacting the cell with a low dose of type I IFN prior to administration of TLR3 ligand.

### BRIEF DESCRIPTION OF THE DRAWING

The foregoing and other features of the present invention will be more readily apparent from the following Detailed Description of the Invention and Brief Description of the Drawing in which:

FIG. 1 is a set of graphs that show the effect of siRNA silencing of TLR3 on apoptosis of Cama-1 cells after incubation for 48 hours with Poly IC.

### DETAILED DESCRIPTION OF THE INVENTION

All publications cited herein are incorporated by reference in their entirety.

Definitions

The term “apoptosis” means programmed cell death.

The term “agonist” means a ligand that is capable of binding to and activating a receptor.

The term “antagonist” means a ligand that is capable of binding to and blocking or inactivating a receptor. Alternatively, an “antagonist” can bind to and block or inactivate an agonist so as to prevent it from binding to a receptor.

The term “antibody” means an entire immunoglobulin, i.e., containing two F_{ab} fragments connected to an F_{c} fragment. The term “antibody” includes polyclonal, monoclonal, chimeric, primatized, humanized and human antibodies. The term “antibody” includes any one of the five major classes of immunoglobulins: IgA, IgD, IgE, IgG and IgM, and also subclasses (isotypes) of immunoglobulins, i.e., IgG1, IgG2, IgG3, IgG4, IgA and IgA2.

The term “antibody fragment” means any fragment or combination of fragments of an entire immunoglobulin, such as, F_{ab}, F_{c}, F_{(ab)2} and Fᵥ fragments.

The term “cancer” describes the physiological condition that is typically characterized by unregulated cell growth. Examples of cancer include, but are not limited to, carcinoma, lymphoma, blastoma, sarcoma and leukemia. More specific examples include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancers.

The term “chemotherapeutic agent” means a chemical compound useful in the treatment of cancer.

The term “treatment” means therapeutic, prophylactic or suppressive measures for a disease or disorder leading to any clinically desirable or beneficial effect, including, but not limited to, alleviation of one or more symptoms, regression, slowing or cessation of progression of the disease or disorder.

The term “siRNA” means short interfering RNA.

The term “TLR” means Toll-like receptor. The TLR can be any species of Toll-like receptor. Preferably, the term refers to a human Toll-like receptor (hTLR), such as one of TLRs 1-10.

The term “TLR expressing cancer” means a tumor containing cells that express a Toll-like receptor.

The term “TLR expressing tumor cell” means a tumor cell that expresses a Toll-like receptor.

The terms “express”, “expresses”, “expression” and “expressing” all mean the transcription and translation of a nucleic acid to produce a polypeptide. In a cell, this means that the polypeptide will either be secreted, remain in the cytoplasm, or reside at least partially in the cell membrane.

The term “ligand” means any molecule that is capable of specifically binding to another molecule, such as a receptor. The term “ligand” includes both agonists and antagonists. A “ligand” can be, for example, a small molecule (an organic molecule), an antibody or antibody fragment, siRNA, an antisense nucleic acid, a polypeptide, DNA and RNA.

The term “TLR ligand” means any molecule capable of specifically binding to a Toll-like receptor, particularly human TLRs 1-10. The term “TLR ligand” includes both agonists and antagonists of TLRs. A “TLR ligand” can be, for example, a small molecule (an organic molecule), an antibody or antibody fragment, siRNA, an antisense nucleic acid, a polypeptide, DNA and RNA.

The term “corresponding TLR ligand” means a ligand that binds to a particular TLR. For example, a TLR1 ligand is the corresponding TLR ligand for TLR1. Likewise, a TLR2 ligand is the corresponding TLR ligand for TLR2. This same principle applies for TLRs 3-10.

The term “patient” means both human and non-human animals.

The term “Poly IC” means polyinosinic-polycytidylic acid.

The term “Poly AU” means polyadenylic-polyuridylic acid.

The term “therapeutically effective amount” means an amount of a composition, such as a TLR ligand, that will ameliorate one or more of the parameters that characterize medical conditions caused or mediated by TLRs, such as cancer.

The terms “effective amount” and “amount effective” mean an amount of a pharmaceutical composition, such as a TLR ligand, that will cause a certain effect, such as the induction of apoptosis in a cell.

The term “low dose” means an amount of a substance that is lower than what is considered normal to achieve a certain effect, such as a therapeutic effect.

Toll-like Receptor (TLR) Characterization

The family of human Toll-like receptors (hTLRs) is comprised of ten members, hTLRs 1-10. The nucleotide sequence of the complete open reading frame and the corresponding amino acid sequence of each of hTLRs 1-10 are known in the art. For example, the sequences for hTLRs 1 -10 are disclosed in PCT Publication No. WO 01/90151, although the sequences are numbered differently than in the public nomenclature. The nucleotide and amino acid sequences for each of hTLRs 1-10 may also be found in the GenBank® database, as shown below in Table 1.

TABLE 1

| | GenBank No. for | GenBank No. for |
|---|---|---|
| TLR | Nucleotide Sequence | Amino Acid Sequence |
| HTLR1 | NM 003263 | NP 003254 |
| HTLR2 | NM 003264 | NP 003255 |
| HTLR3 | NM 003265 | NP 003256 |
| HTLR4 | NM 138557 (isoform 4) | NP 612567 (isoform D) |
| | NM 138556 (isoform 2) | NP 612566 (isoform B) |
| | NM 138554 (isoform 1) | NP 612564 (isoform A) |
| | NM 003266 (isoform 3) | NP 003257 (isoform C) |
| HTLR5 | NM 003268 | NP 003259 |
| HTLR6 | NM 006068 | NP 006059 |
| HTLR7 | NM 016562 | NP 057646 |
| HTLR8 | NM 138636 (isoform 2) | NP 619542 (isoform 2) |
| | NM 016610 (isoform 1) | NP 057694 (isoform 1) |
| HTLR9 | NM 138688 (isoform B) | NP 619633 (isoform B) |
| | NM 017442 (isoform A) | AAF72189 (isoform A) |
| hTLR10 | NM 030956 | AAK26744 |

A person having skill in the art will, given the nucleic acid and amino acid sequence of any TLR, be able to produce any TLR protein or fragment thereof, antibody to the protein or fragment, nucleic acid or fragment thereof, nucleic acid probe, antisense, siRNA, etc. using standard molecular biology techniques. These molecules can then be used to select a TLR expressing cancer or tumor cell.

Some TLR ligands have been identified, as shown below in Table 2. A person having skill in the art will be able to isolate or generate any of the below ligands. Alternatively, the ligands may be purchased from commercial sources.

TABLE 2

| TLR | Ligands |
|---|---|
| TLR1 | Mycoplasma lipopeptides (diacylated lipoproteins) (Sigma- |
| | Aldrich) |
| TLR2 | Mycoplasma lipopeptides (diacylated lipoproteins) (Sigma- |
| | Aldrich), bacterial lipopeptides (Sigma-Aldrich) |
| TLR3 | dsRNA (Invivogen), polyadenylic-polyuridylic acid (Poly AU) |
| | (Invivogen), polyinosinic-polycytidylic acid (Poly IC) |
| | (Invivogen) |
| TLR4 | LPS (Sigma-Aldrich) |
| TLR5 | Flagellin (Calbiochem) |
| TLR6 | Bacterial lipopeptides (Sigma-Aldrich) |
| TLR7 | Imiquimod (Aldara ®) (3M Pharmaceuticals), |
| | R848 (3M Pharmaceuticals) |
| TLR8 | R848 (3M Pharmaceuticals) |
| TLR9 | CpG DNA (MWG Biotech) |
| TLR10 | Unknown |

TLRs function as mediators of the immune response. Therefore, therapeutic applications for TLRs exist in the areas of oncology, infectious disease, autoimmunity, allergy, asthma, COPD and cardiology.

The present invention is based, in part, on the discovery that certain types of tumor cells express Toll-like receptors and that ligand binding to these TLRs help in the establishment and improve the effectiveness of tumor directed immune responses.

Selecting a TLR Expressing Cancer or Tumor Cell

A step of the method of the invention involves selecting a patient that has a TLR expressing cancer or selecting a TLR expressing tumor cell.

The term “selecting” means to identify something of interest. In the context of the present application, the phrase “selecting a patient” means to identify a patient having a particular characteristic, such as a TLR expressing cancer. The phrase “selecting a TLR expressing tumor cell” means to identify a tumor cell that expresses a Toll-like receptor.

As is known in the art, there are many ways of selecting a patient that has a TLR expressing cancer or selecting a TLR expressing tumor cell. For example, an antibody or an antibody fragment may be used to bind to and identify a TLR expressing tumor cell. Preferably, a TLR3 antibody is used to bind to and identify a TLR3 expressing tumor cell. The antibody or fragment thereof may be given in vivo in a pharmaceutical composition or in vitro. Preferably, a biopsy is performed on a patient and the tumor cells are selected in vitro. It is also possible to increase the expression of the TLR before the biopsy as a potential means of recruiting patients that would otherwise not have been included in the protocol for TLR ligand treatment. In the case of TLR3, a low dose of type I IFN or TLR3 ligand itself might be administered for a few days before biopsy or before any other diagnostic procedure (needle aspiration or medical imagery). Alternatively, any one of the TLR ligands identified in Table 2 of this application, or other small molecules may be used to bind to and identify a TLR expressing tumor cell. Preferably, a TLR3 ligand is used to bind to and identify a TLR3 expressing cell. Again, the selecting step is preferably performed in vitro. Furthermore, tumor cells may be lysed to determine whether the cells exhibit increased levels of a particular TLR protein (by Western blot) or a particular TLR RNA (by Northern blot).

The selecting process may involve the use of detectable labels. For example, the above antibodies, antibody fragments, small molecules, DNA, RNA, and other ligands may need to be labeled in order to be detected. Detection may be accomplished visually, or by the use of a device. Detectable labels commonly used in the art include, for example, radiolabels, fluorescent labels, and enzymatic labels, although any detectable label can be used.

In addition to identifying a tumor cell that expresses a TLR, the selecting step will probably identify which Toll-like receptor (TLRs 1-10) a particular tumor cell is expressing. This is due to the fact that many antibodies, antibody fragments, DNAs, RNAs, small molecules, or other ligands used for selecting a TLR expressing tumor cell specifically binds to an individual TLR of TLRs 1-10.

The step of selecting a patient that has a TLR expressing cancer or selecting a TLR expressing tumor cell can also be performed in an indirect manner. For example, the expression of a particular TLR by a cancer may be linked to a specific sub-type of cancer with a specific etiology. Any marker of this specific etiology, such as a virus, may be indicative of the expression of a given TLR and may be a useful marker for guiding the use of the corresponding TLR ligand.

Administering TLR Ligands to Patients

Another step of the method of the invention involves administering to a patient a therapeutically effective amount of a TLR ligand. This step involves administering the TLR ligand in a pharmaceutical composition. For example, the pharmaceutical composition may be in the form of a tablet, such that the ligand is absorbed into the bloodstream. The circulatory system can then deliver the TLR ligand to a TLR expressing cancer such that the ligand and the cancer may contact each other. This contacting step will allow the ligand to bind to the cancer's Toll-like receptor(s) and induce growth inhibition and apoptosis in the cancer. Alternatively, the pharmaceutical composition may be administered locally or topically, such as for the treatment of melanoma.

As stated above, the selecting step will probably identify the particular TLR that the cancer is expressing. Preferably, the administering step involves administering a corresponding ligand to a patient having a cancer that expresses a Toll-like receptor. For example, if a cancer expresses TLR1, the patient is preferably administered an effective amount of a TLR1 ligand. Likewise, if a cancer expresses TLR2, the patient is preferably administered an effective amount of a TLR2 ligand. The same principle holds true for TLRs 3-10.

Preferably, the method of the invention involves administering to a patient having a TLR3 expressing cancer an effective amount of a TLR3 ligand. Preferably, the TLR3 ligand is an agonist. More preferably, the TLR3 ligand is Poly AU. Most preferably, the TLR3 ligand is Poly IC. Preferably, the cancer is colon cancer cell or breast cancer.

Preferably, the method of the invention further comprises administering to the patient a chemotherapeutic agent or a cancer treatment.

Preferably, the method of the invention further comprises administering to the patient a low dose of type I IFN or TLR3 ligand. For example, a low dose of type I IFN is in the range of 1-3 MU, and preferably 2 MU. More preferably, the low dose of type I IFN is less than 1 MU.

Contacting TLR Expressing Tumor Cells with TLR Ligands

Alternatively, a step of the method of the invention involves contacting a TLR expressing tumor cell with an effective amount of a TLR ligand. In vivo, the contacting step involves administering a TLR ligand in a pharmaceutical composition to a patient. In vitro, the contacting step involves bringing a TLR expressing tumor cell and TLR ligand into close physical proximity such that the ligand and the cell may contact each other. This contacting step will allow the ligand to bind to the cell's Toll-like receptor and induce growth inhibition and apoptosis in the tumor cell.

As stated above, the selecting step will probably identify the particular TLR that the tumor cell is expressing. Preferably, the contacting step involves contacting a cell that expresses a Toll-like receptor to its corresponding ligand. For example, if a tumor cell expresses TLR1, the cell is preferably contacted with an effective amount of a TLR1 ligand. Likewise, if a tumor cell expresses TLR2, the cell is preferably contacted with an effective amount of a TLR2 ligand. The same principle holds true for TLRs 3-10.

Preferably, the method of the invention involves contacting a TLR3 expressing tumor cell with an effective amount of a TLR3 ligand. Preferably, the TLR3 ligand is an agonist. More preferably, the TLR3 ligand is Poly AU. Most preferably, the TLR3 ligand is Poly IC. Preferably, the cell is a colon cancer cell or a breast cancer cell.

Preferably, the method of the invention further comprises contacting the cell with a chemotherapeutic agent or a cancer treatment.

Preferably, the method of the invention further comprises contacting the cell with a low dose of type I IFN or TLR3 ligand. For example, a low dose of type I IFN is in the range of 1-3 MU, and preferably 2 MU. More preferably, the low dose of type I IFN is less than 1 MU.

Polypeptides

Polypeptides, such as an antibody, an antibody fragment or a lipopeptide, may be used in the selecting step, to select a TLR expressing cancer or cell, in the administering step, to deliver a TLR ligand to a patient, or in the contacting step, to induce growth inhibition and apoptosis in a TLR expressing cell, in the method of the present invention. In addition, TLR polypeptides or fragments thereof can be produced in order to identify or generate ligands, such as an antibody, that will bind to the TLR.

As used herein, the term “polypeptide” or “peptide” means a fragment or segment, e.g., of a polypeptide containing at least 8, preferably at least 12, more preferably at least 20, and most preferably at least 30 or more contiguous amino acid residues, up to and including the total number of residues in the complete protein. The term “polypeptide” also encompasses deletions, additions, modifications, substitutions, analogs, variants, and glycosylated or non-glycosylated polypeptides.

Substitutions include both conservative and non-conservative substitutions.

Modifications of amino acid residues may include, but are not limited to, aliphatic esters or amides of the carboxyl terminus or of residues containing carboxyl side chains, O-acyl derivatives of hydroxyl group-containing residues, and N-acyl derivatives of the amino-terminal amino acid or amino-group containing residues, e.g., lysine or arginine.

Analogs are polypeptides containing modifications, such as incorporation of unnatural amino acid residues, or phosphorylated amino acid residues, such as phosphotyrosine, phosphoserine or phosphothreonine residues. Other potential modifications include sulfonation, biotinylation, or the addition of other moieties, particularly those that have molecular shapes similar to phosphate groups.

Techniques for the synthesis of polypeptides are described, for example, in Merrifield, J. Amer. Chem. Soc. 85:2149 (1963); Merrifield, Science 232:341 (1986); and Atherton et al., Solid Phase Peptide Synthesis: A Practical Approach, 1989, IRL Press, Oxford.

Analogs of polypeptides can be prepared by chemical synthesis or by using site-directed mutagenesis [Gillman et al., Gene 8:81 (1979); Roberts et al., Nature, 328:731 (1987) or Innis (Ed.), 1990, PCR Protocols: A Guide to Methods and Applications, Academic Press, New York, N.Y.] or the polymerase chain reaction method [PCR; Saiki et al., Science 239:487 (1988)], as exemplified by Daugherty et al. [Nucleic Acids Res. 19:2471 (1991)] to modify nucleic acids encoding the complete receptors. Adding epitope tags for purification or detection of recombinant products is envisioned.

Nucleic Acids

Nucleic acids may be used for selecting a patient having a TLR expressing cancer or for selecting a TLR expressing tumor cell. In order to select a patient, a biopsy of the patient's tumor is preferably performed. Then, the tumor cells can be analyzed in vitro for expression of TLR nucleic acids.

As shown in Table 1 of this application, the nucleic acid and amino acid sequences of each of hTLRs 1-10 are known in the art. One having skill in the art is able to use the known sequences or fragments thereof in order to generate a hybridization assay to determine whether a particular tumor cell is expressing TLR nucleic acids. For example, using the known sequence for a particular TLR, a person having skill in the art could perform a Northern blot analysis to determine whether a tumor cell is expressing that particular TLR.

In addition, nucleic acids encoding specific TLRs or fragments thereof may be used to generate TLR polypeptides. The TLR polypeptides can then be used to generate antibodies to a specific TLR.

A nucleic acid “fragment” is defined herein as a nucleotide sequence comprising at least 17, generally at least 25, preferably at least 35, more preferably at least 45, and most preferably at least 55 or more contiguous nucleotides.

General techniques for nucleic acid manipulation and expression are described generally, e.g., in Sambrook, et al., Molecular Cloning: A Laboratory Manual (2d ed.), 1989, Vols. 1-3, Cold Spring Harbor Laboratory.

Antibody Production

Antibodies and fragments thereof that are specific for TLRs may be used in either the selecting step, for selecting a TLR expressing cell, in the administering step, to deliver a TLR ligand to a patient, or in the contacting step, to induce growth inhibition and apoptosis in a TLR expressing cell, of the method of the present invention.

Antigenic (i.e., immunogenic) fragments of an individual TLR may be produced. Regardless of whether they bind the TLR ligands, such fragments, like the complete receptors, are useful as antigens for preparing antibodies that can bind to the complete receptors. Shorter fragments can be concatenated or attached to a carrier. Because it is well known in the art that epitopes generally contain at least about five, preferably at least 8, amino acid residues [Ohno et al., Proc. Natl. Acad. Sci. USA 82:2945 (1985)], fragments used for the production of antibodies will generally be at least that size. Preferably, they will contain even more residues, as described above. Whether a given fragment is immunogenic can readily be determined by routine experimentation.

Although it is generally not necessary when complete TLRs are used as antigens to elicit antibody production in an immunologically competent host, smaller antigenic fragments are preferably first rendered more immunogenic by cross-linking or concatenation, or by coupling to an immunogenic carrier molecule (i.e., a macromolecule having the property of independently eliciting an immunological response in a host animal). Cross-linking or conjugation to a carrier molecule may be required because small polypeptide fragments sometimes act as haptens (molecules that are capable of specifically binding to an antibody but incapable of eliciting antibody production, i.e., they are not immunogenic). Conjugation of such fragments to an immunogenic carrier molecule renders them more immunogenic through what is commonly known as the “carrier effect”.

Suitable carrier molecules include, e.g., proteins and natural or synthetic polymeric compounds, such as polypeptides, polysaccharides, lipopolysaccharides, etc. Protein carrier molecules are especially preferred, including, but not limited to, keyhole limpet hemocyanin and mammalian serum proteins, such as human or bovine gammaglobulin, human, bovine or rabbit serum albumin, or methylated or other derivatives of such proteins. Other protein carriers will be apparent to those skilled in the art. Preferably, but not necessarily, the protein carrier will be foreign to the host animal in which antibodies against the fragments are to be elicited.

Covalent coupling to the carrier molecule can be achieved using methods well known in the art, the exact choice of which will be dictated by the nature of the carrier molecule used. When the immunogenic carrier molecule is a protein, the fragments of the invention can be coupled, e.g., using water-soluble carbodiimides, such as dicyclohexylcarbodiimide or glutaraldehyde.

Coupling agents such as these can also be used to cross-link the fragments to themselves without the use of a separate carrier molecule. Such cross-linking into aggregates can also increase immunogenicity. Immunogenicity can also be increased by the use of known adjuvants, alone or in combination with coupling or aggregation.

Suitable adjuvants for the vaccination of animals include, but are not limited to, Adjuvant 65 (containing peanut oil, mannide monooleate and aluminum monostearate); Freund's complete or incomplete adjuvant; mineral gels, such as aluminum hydroxide, aluminum phosphate and alum; surfactants, such as hexadecylamine, octadecylamine, lysolecithin, dimethyldioctadecylammonium bromide, N,N-dioctadecyl-N′,N′-bis(2-hydroxymethyl)propanediamine, methoxyhexadecylglycerol and pluronic polyols; polyanions, such as pyran, dextran sulfate, poly IC, polyacrylic acid and carbopol; peptides, such as muramyl dipeptide, dimethylglycine and tuftsin; and oil emulsions. The polypeptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition, 1987, Elsevier, New York. Other useful references covering methods for preparing polyclonal antisera include Microbiology, 1969, Hoeber Medical Division, Harper and Row; Landsteiner, Specificity of Serological Reactions, 1962, Dover Publications, New York, and Williams, et al., Methods in Immunology and Immunochemistry, Vol. 1, 1967, Academic Press, New York.

Serum produced from animals immunized using standard methods can be used directly, or the IgG fraction can be separated from the serum using standard methods, such as plasmaphoresis or adsorption chromatography with IgG-specific adsorbents, such as immobilized Protein A. Alternatively, monoclonal antibodies can be prepared.

Hybridomas producing monoclonal antibodies against the TLRs or antigenic fragments thereof are produced by well-known techniques. Usually, the process involves the fusion of an immortalizing cell line with a B-lymphocyte that produces the desired antibody. Alternatively, non-fusion techniques for generating immortal antibody-producing cell lines can be used, e.g., virally-induced transformation [Casali et al., Science 234:476 (1986)]. Immortalizing cell lines are usually transformed mammalian cells, particularly myeloma cells of rodent, bovine, and human origin. Most frequently, rat or mouse myeloma cell lines are employed as a matter of convenience and availability.

Techniques for obtaining antibody-producing lymphocytes from mammals injected with antigens are well known. Generally, peripheral blood lymphocytes (PBLs) are used if cells of human origin are employed, or spleen or lymph node cells are used from non-human mammalian sources. A host animal is injected with repeated dosages of the purified antigen (human cells are sensitized in vitro), and the animal is permitted to generate the desired antibody-producing cells before they are harvested for fusion with the immortalizing cell line. Techniques for fusion are also well known in the art, and in general involve mixing the cells with a fusing agent, such as polyethylene glycol.

Hybridomas are selected by standard procedures, such as HAT (hypoxanthine-aminopterin-thymidine) selection. Those secreting the desired antibody are selected using standard immunoassays, such as Western blotting, ELISA (enzyme-linked immunosorbent assay), RIA (radioimmunoassay), or the like. Antibodies are recovered from the medium using standard protein purification techniques [Tijssen, Practice and Theory of Enzyme Immunoassays (Elsevier, Amsterdam, 1985)].

Many references are available to provide guidance in applying the above techniques [Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982)]. Monoclonal antibodies can also be produced using well-known phage library systems. See, e.g., Huse, et al., Science 246:1275 (1989); Ward, et al., Nature, 341:544 (1989).

Antibodies thus produced, whether polyclonal or monoclonal, can be used, e.g., in an immobilized form bound to a solid support by well known methods, to purify the receptors by immunoaffinity chromatography.

Antibodies against the antigenic fragments can also be used, unlabeled or labeled by standard methods, as the basis for immunoassays of the TLRs. The particular label used will depend upon the type of immunoassay. Examples of labels that can be used include, but are not limited to, radiolabels, such as ³²P, ¹²⁵I, ³H and ¹⁴C; fluorescent labels, such as fluorescein and its derivatives, rhodamine and its derivatives, dansyl and umbelliferone; chemiluminescers, such as luciferia and 2,3-dihydrophthalazinediones; and enzymes, such as horseradish peroxidase, alkaline phosphatase, lysozyme and glucose-6-phosphate dehydrogenase.

The antibodies can be tagged with such labels by known methods. For example, coupling agents, such as aldehydes, carbodiimides, dimaleimide, imidates, succinimides, bisdiazotized benzadine and the like may be used to tag the antibodies with fluorescent, chemiluminescent or enzyme labels. The general methods involved are well known in the art and are described, e.g., in Immunoassay: A Practical Guide, 1987, Chan (Ed.), Academic Press, Inc., Orlando, Fla. Such immunoassays could be carried out, for example, on fractions obtained during purification of the receptors.

The antibodies of the present invention can also be used to identify particular cDNA clones expressing the TLRs in expression cloning systems.

Neutralizing antibodies specific for the ligand-binding site of a receptor can also be used as antagonists (inhibitors) to block ligand binding. Such neutralizing antibodies can readily be identified through routine experimentation, e.g., by using the radioligand binding assay described infra. Antagonism of TLR activity can be accomplished using complete antibody molecules, or well-known antigen binding fragments such as Fab, Fc, F(ab)₂, and Fv fragments.

Definitions of such fragments can be found, e.g., in Klein, Immunology (John Wiley, New York, 1982); Parham, Chapter 14, in Weir, ed. Immunochemistry, 4th Ed. (Blackwell Scientific Publishers, Oxford, 1986). The use and generation of antibody fragments has also been described, e.g.: Fab fragments [Tijssen, Practice and Theory of Enzyme Immunoassays (Elsevier, Amsterdam, 1985)], Fvfragments [Hochman et al., Biochemistry 12:1130 (1973); Sharon et al., Biochemistry 15:1591 (1976); Ehrlich et al., U.S. Pat. No. 4,355,023] and antibody half molecules (Auditore-Hargreaves, U.S. Pat. No. 4,470,925). Methods for making recombinant Fv fragments based on known antibody heavy and light chain variable region sequences have further been described, e.g., by Moore et al. (U.S. Pat. No. 4,642,334) and by Plückthun [Bio/Technology 9:545 (1991)]. Alternatively, they can be chemically synthesized by standard methods.

Anti-idiotypic antibodies, both polyclonal and monoclonal, can also be produced using the antibodies elicited against the receptors as antigens. Such antibodies can be useful as they may mimic the receptors.

Pharmaceutical Compositions

TLR agonists and antagonists can be used therapeutically to stimulate or block the activity of a TLR, and thereby to treat any medical condition caused or mediated by the TLR. The dosage regimen involved in a therapeutic application will be determined by the attending physician, considering various factors which may modify the action of the therapeutic substance, e.g., the condition, body weight, sex and diet of the patient, time of administration, and other clinical factors.

Typical protocols for the therapeutic administration of such substances are well known in the art. Administration of the pharmaceutical compositions is typically by parenteral, intraperitoneal, intravenous, subcutaneous, or intramuscular injection, or by infusion or by any other acceptable systemic method. Often, treatment dosages are titrated upward from a low level to optimize safety and efficacy. Generally, daily dosages will fall within a range of about 0.01 to 20 mg protein per kilogram of body weight. Typically, the dosage range will be from about 0.1 to 5 mg per kilogram of body weight.

Dosages will be adjusted to account for the smaller molecular sizes and possibly decreased half-lives (clearance times) following administration. It will be appreciated by those skilled in the art, however, that the TLR antagonists encompass neutralizing antibodies or binding fragments thereof in addition to other types of inhibitors, including small organic molecules and inhibitory ligand analogs, which can be identified using the methods of the invention.

Although the pharmaceutical compositions could be administered in simple solution, they are more typically used in combination with other materials such as carriers, preferably pharmaceutical carriers. Useful pharmaceutical carriers can be any compatible, non-toxic substances suitable for delivering the pharmaceutical compositions to a patient. Sterile water, alcohol, fats, waxes, and inert solids may be included in a carrier. Pharmaceutically acceptable adjuvants (buffering agents, dispersing agents) may also be incorporated into the pharmaceutical composition. Generally, compositions useful for parenteral administration of such drugs are well known, e.g. Remington's Pharmaceutical Science, 17th Ed. (Mack Publishing Company, Easton, Pa., 1990). Alternatively, pharmaceutical compositions may be introduced into a patient's body by implantable drug delivery systems [Urquhart et al., Ann. Rev. Pharmacol. Toxicol. 24:199 (1984)].

Therapeutic formulations may be administered in many conventional dosage formulations. Formulations typically comprise at least one active ingredient, together with one or more pharmaceutically acceptable carriers. Formulations may include those suitable for oral, rectal, nasal or parenteral (including subcutaneous, intramuscular, intravenous and intradermal) administration.

The formulations may conveniently be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

Combination Therapies

The effectiveness of a TLR ligand in preventing or treating cancer may be improved by administering the ligand in combination with another agent or treatment that is effective for the same purpose. For example, a TLR ligand may be administered in combination with a chemotherapeutic agent or a cancer treatment. Preferably, the TLR ligand is a TLR3 agonist.

A “chemotherapeutic agent” is a chemical compound useful in the treatment of cancer. Examples of chemotherapeutic agents include alkylating agents, such as thiotepa and cyclosphosphamide (CYTOXAN™); alkyl sulfonates, such as busulfan, improsulfan and piposulfan; aziridines, such as benzodopa, carboquone, meturedopa and uredopa; ethylenimines and methylamelamines, including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and uracil mustard; nitrosureas, such as carmustine, chlorozotocin, fotemustine, lomustine, nimustine, ranimustine; antibiotics, such as the enediyne antibiotics (e.g. calicheamicin, especially calicheamicin gamma1I and calicheamicin phil1, see, e.g., Agnew, Chem Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores, aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin and zorubicin; anti-metabolites, such as methotrexate and 5-fluorouracil (5-FU); folic acid analogues such as denopterin, methotrexate, pteropterin and trimetrexate; purine analogs, such as fludarabine, 6-mercaptopurine, thiamiprine and thioguanine; pyrimidine analogs, such as ancitabine, azacitidine, 6-azauridine, carmofur, cytarabine, dideoxyuridine, doxifluridine, enocitabine and floxuridine; androgens, such as calusterone, dromostanolone propionate, epitiostanol, mepitiostane and testolactone; anti-adrenals, such as aminoglutethimide, mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids, such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogs, such as cisplatin and carboplatin; vinblastine; platinum; etoposide (VP-16); ifosfamide; mitoxantrone; vincristine; vinorelbine (NavelbineT™); novantrone; teniposide; edatrexate; daunomycin; aminopterin; xeloda; ibandronate; CPT-11; topoisomerase inhibitor RFS 2000; difluoromethylornithine (DMFO); retinoids, such as retinoic acid; capecitabine; and pharmaceutically acceptable salts, acids or derivatives of any of the above. Also included in this definition are anti-hormonal agents that act to regulate or inhibit hormone action on tumors, such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens, such as flutamide, nilutamide, bicalutamide, leuprolide and goserelin; and pharmaceutically acceptable salts, acids or derivatives of any of the above.

A “treatment” for cancer includes surgery, to remove a cancer, and radiation treatment, to reduce or kill a cancer or tumor. The effectiveness of a TLR ligand in preventing or treating cancer may also be improved by administering the ligand in combination with a low dose of type I IFN. For example, a low dose of type I IFN is in the range of 1-3 MU, and preferably 2 MU. More preferably, the low dose of type I IFN is less than 1 MU. Preferably, the TLR ligand is a TLR3 agonist.

As stated above, the dosage regimen involved in a combination therapy will be determined by the attending physician.

### EXAMPLES

The present invention may be better understood by reference to the following non-limiting examples, which are provided as exemplary of the invention. The following examples are presented in order to more fully illustrate the invention, and should in no way be construed as limiting the broad scope of the invention. Unless otherwise indicated, percentages given below for solids in solid mixtures, liquids in liquids, and solids in liquids are on a wt/wt, vol/vol and wt/vol basis, respectively. Sterile conditions were generally maintained during cell culture.

### Materials and General Methods

Standard methods were used, as described, e.g., in Maniatis et al., Molecular Cloning: A Laboratory Manual, 1982, Cold Spring Harbor Laboratory, Cold Spring Harbor Press; Sambrook et al., Molecular Cloning: A Laboratory Manual, (2d ed.), Vols 1-3, 1989, Cold Spring Harbor Press, NY; Ausubel et al., Biology, Greene Publishing Associates, Brooklyn, N.Y.; or Ausubel, et al. (1987 and Supplements), Current Protocols in Molecular Biology, Greene/Wiley, New York; Innis et al. (eds.) PCR Protocols: A Guide to Methods and Applications, 1990, Academic Press, N.Y.

Cell Lines and Reagents

Human breast tumor cell lines, Cama-1, SW527, BT-483 and MCF-7, were obtained from the ATCC (Rockville, Md.) and cultured in DMEM F12 containing 4.5 g/mL glucose (Invitrogen, San Diego, Calif.) complemented with 2 mM L-glutamine (Life Technologies, Paisley Park, GB), 10% fetal calf serum (Life Technologies), 160 μg/mL gentalline (Schering Plough, Kenilworth, N.J.), 2.5 mg/mL sodium bicarbonate (Life Technologies), amino acids (Invitrogen) and 1 mM sodium pyruvate (Sigma-Aldrich, Saint Louis, Mo.) (referred to as complete medium). Polyinosinic-polycytidilic acid, Poly IC, was obtained from Invivogen (San Diego, Calif.). Peptidoglycan (PGN) and lipopolysaccharide (LPS) were purchased from Sigma-Aldrich. Type I IFN receptor blocking mAb was purchased from PBL Biochemical Laboratories (Piscataway, N.J.) and TNF-α neutralizing mAb was purchased from Genzyme (Cambridge, Mass.). Antibodies to Stat1, phosphorylated Stat1 (tyrosine 701) and PKR were purchased from Cell Signaling (Beverly, Mass.). Antibodies to human IFN-β were purchased from R&D Systems (Minneapolis, Minn.). Antibodies to NF-κB p65 subunit, TRAF6 and β-tubulin were purchased from Santa Cruz Biotechnology (Santa Cruz, Calif.). The general caspase inhibitor z-VAD-fmk was purchased from R&D Systems. Cycloheximide (CHX) was purchased from Sigma-Aldrich.

Human primary breast tumor sample was obtained from the Centre Léon Bérard (Lyon, France) in agreement with the Hospital bioethical protocols. A single cell suspension was obtained after digestion with Collagenase A (Sigma-Aldrich) and washes and enrichment in Human Epithelial Antigen (HEA) positive cells using HEA-microbeads (Mylteni Biotech, Bergisch Gladbach, Germany) according to manufacturer's instructions. The final single cell suspension contained more than 80% HEA positive cells and less than 2% CD4⁺ hematopoietic contaminants.

Apoptosis Analysis

Cell recovery after treatment with TLR ligands was measured by crystal violet staining (Sigma-Aldrich). Cells were plated at 10⁴ cells/well in 96 well plates. After 72 hours of culture either with or without TLR ligand, the cells were washed with PBS, fixed in 6% formaldehyde (Sigma-Aldrich) for 20 minutes, washed twice, and then stained with 0.1% crystal violet for 10 minutes. After washes and incubation in 1% SDS for 1 hour, the absorbance was read at 605 nm on a Vmax plate reader (Molecular Devices, Sunnyvale, Calif.). Annexin V staining was performed with an annexin-FITC apoptosis detection kit (BD Pharmingen, San Diego, Calif.) according to the manufacturer's instructions. Sub-diploid cells were detected by staining with 3 μg/mL propidium iodide (PI) (Molecular probes, Eugene, Oreg.), after overnight permeabilization in 70% ethanol. Fluorescence was analyzed by flow cytometry on a FACScalibur (Becton Dickinson, Mountain View, Calif.) equipped with a doublet-discrimination module and using Celiquest Pro software (Becton Dickinson).

Biochemistry

Cama-1 cells were lysed in 1% Nonidet-P40-containing buffer. 20 μg total protein was loaded per lane on SDS-Polyacrylamide gels (Invitrogen). Western Blots (WB) were performed with standard techniques using the antibodies described above. Anti IRAK-4 monoclonal antibodies were generated in the laboratory according to the protocol described in Fossiez et al., “T cell interleukin-17 induces stromal cells to produce proinflammatory and hematopoietic cytokines”, J. Exp. Med., vol. 183(6), pp. 2593-2603 (1996).

Cytokine Secretion

IL-6 secretion was measured in culture supernatants by standard Enzyme-Linked Assay (ELISA) using a DuoSet ELISA kit according to manufacturer's instructions (R&D Systems).

siRNA Experiments

Cama-1 cells were plated in 6 well plates at 3×10⁵ cells per well. After overnight adherence, siRNA transfections were performed for 5 hours in OptiMEM medium (Life technologies) containing 3 μg/mL lipofectamine 2000 (Invivogen) and 100 nM siRNA. Cells were then washed and cultured for 72 hours in complete medium before treatment with Poly IC and apoptosis analysis. siRNA duplexes specific for TLR3, PKR, IRAK-4, TRAF6 and p65 were purchased from Dharmacon (Lafayette, Colo.) as SMART-Pools. TRIF siRNA was purchased from the same supplier as single oligoduplexes (5′-GCUCUUGUAUCUGMGCAC-3′) (SEQ ID NO: 23). TLR3 and TRIF expression was assessed by PCR (35 cycles: 1 min 94° C., 1 min 55° C., 2 min 72° C.) with Taq PCR ReadyMix (Sigma-Aldrich) using following primers:

(SEQ ID NO: 24)

5′-AACGATTCCTTTGCTTGGCTTC-3′ (forward)/

(SEQ ID NO: 25)

5′-GCTTAGATCCAGAATGGTCAAG-3′ (reverse)
for TLR3
and

(SEQ ID NO: 26)

5′-ACTTCCTAGCGCCTTCGACA-3′ (forward)/

(SEQ ID NO: 27)

5′-ATCTTCTACAGAAAGTTGGA-3′ (reverse)
for TRIF.

Expression of PKR, IRAK4, TRAF6 and p65 was assessed by WB as described above.

### Example 1

In these sets of experiments, TLR expression for each of TLRs 1-10 was detected with RT-PCR in six human colorectal adenocarcinoma cell lines. The six cell lines analyzed were Caco 2, LoVo, Colo 320 DM, SNU-C1, T84 and Colo 205. Equal amounts of mRNA were extracted from each cell line. The mRNA was subsequently amplified by PCR for 35 cycles (30 sec. at 94° C., 45 sec. at 60° C., 90 sec. at 72° C.) using hTLR-specific primers. The following primers were used:

| | | |
|---|---|---|
| TLR1F: | caggatcaaggtacttgatcttc; | (SEQ ID NO: 1) |
| TLR1R: | tttctctcatgaaggcaaatctg; | (SEQ ID NO: 2) |
| TLR2F: | ctcaggagcagcaagcactg; | (SEQ ID NO: 3) |
| TLR2R: | atcttccgcagcttgcagaag; | (SEQ ID NO: 4) |
| TLR3F: | aacgattcctttgcttggcttc; | (SEQ ID NO: 5) |
| TLR3R: | gcttagatccagaatggtcaag; | (SEQ ID NO: 6) |
| TLR4F: | ctcagaatgactttgcttgtac; | (SEQ ID NO: 7) |
| TLR4R: | gcaggacaatgaagatgatacc; | (SEQ ID NO: 8) |
| TLR5F: | cgaacctcatccacttatcag; | (SEQ ID NO: 9) |
| TLR5R: | gtgaactttagggactttaagac; | (SEQ ID NO: 10) |
| TLR6F: | ccaatgtacctgtgagctaag; | (SEQ ID NO: 11) |
| TLR6R: | ccactcactctggacaaagttg; | (SEQ ID NO: 12) |
| TLR7F: | ggatctgtctttcaattttgaac; | (SEQ ID NO: 13) |
| TLR7R: | ccaaggtctgcccatacttg; | (SEQ ID NO: 14) |
| TLR8F: | gctatccttgtgatgagaaaaag; | (SEQ ID NO: 15) |
| TLR8R: | gcattgaagcacctcggacag; | (SEQ ID NO: 16) |
| TLR9F: | actgtttcgccctctcgctg; | (SEQ ID NO: 17) |
| TLR9R: | gccagcacaaacagcgtcttg, | (SEQ ID NO: 18) |
| TLR10F: | ttgttcagagctgccaggaag, | (SEQ ID NO: 19) |
| and | | |
| TLR10R: | gcaaagtagaattcataatggcac. | (SEQ ID NO: 20) |

The PCR products were then analyzed on an agarose gel that was stained with Ethidium Bromide.

The results of these experiments show that the Caco 2 cell line expressed TLRs 2, 5, 7 and 9. The LoVo cell line expressed TLRs 2, 3, 4, 5 and 6. The Colo 320 DM cell line expressed TLRs 5 and 6. The SNU-C1 cell line expressed TLR 4. The T84 cell line expressed TLRs 4, 5 and 6. The Colo 205 cell line expressed TLRs 4, 5 and 6.

A similar analysis was performed on eight human lung cell lines (NCI-H526, SHP-77, NCI-N417, A549, NCI-H358, A427, NCI-H292, NCI-H187) and four human breast cancer cell lines (SW527, Cama-1, BT483, MCF-7). The results of these experiments show that the NCI-H526 cell line (small cell lung carcinoma) expressed TLRs 2, 3, 5 and 9. The SHP-77 cell line (large cell variant of SCLC) expressed TLRs 4, 5, 6, 7, 9 and 10. The NCI-N417 cell line (small cell lung carcinoma) expressed TLR 5. The A549 cell line (lung carcinoma) expressed TLRs 1, 2, 3, 4, 5, 6, 7 and 10. The NCI-H358 cell line (bronchioloalveolar carcinoma) expressed TLRs 2, 4, 5, 6, 7 and 10. The A427 cell line (lung carcinoma) cell line expressed TLRs 2, 3, 5 and 6. The NCI-H292 cell line (epidermoid lung carcinoma) expressed TLRs 1, 2, 3, 4, 5, 6 and 10. The NCI-H187 cell line (small cell lung carcinoma) expressed TLRs 5, 6 and 10. The SW527 cell line (breast adenocarcinoma) expressed TLRs 2, 4, 6 and 10. The Cama-1 cell line (breast adenocarcinoma) expressed TLRs 2, 5, 6 and 10. The BT483 cell line (breast adenocarcinoma) expressed TLRs 2, 4, 5, 6, 7, 9 and 10. The MCF-7 cell line (breast adenocarcinoma) expressed TLRs 2, 5, 6 and 9.

It is apparent that all of the tested human tumor lines from colon, breast and lung express a number of TLR transcripts. However, substantial heterogeneity exists as to which TLRs are expressed in each cell line and to their level of expression.

### Example 2

Four human breast tumor cell lines, Cama-1, SW527, BT483 and MCF-7, were analyzed for cell death in response to Poly IC. Cells were cultured for 72 hours with 5 μg/ml PGN, 50 μg/ml Poly IC or 10 μg/ml LPS. Control cells were cultured with PBS. Cytotoxicity was assessed by crystal violet staining and expressed as a percent of control.

On average, the control cells exhibited 100% cell recovery. The PGN cells exhibited an average of 95% cell recovery. The LPS treated cells exhibited 95% recovery, on average. On average, the cells treated with Poly IC exhibited 67.5% cell recovery. Specifically, The Cama-1, SW527, BT483 and MCF-7 cell lines exhibited cell recoveries of 33%, 75%, 67% and 100%, respectively.

The data show that Poly IC triggered a decrease in cell recovery in three of the cell lines tested, Cama-1, BT483 and SW527. As can be seen from the data, the Cama-1 cell line consistently exhibited the most dramatic reduction. However, Poly IC did not cause a decrease in cell recovery in the MCF-7 cell line.

Furthermore, additional TLR ligands were tested to determine any possible effects on cellular toxicity. The ligands tested were PGN, LPS, Flagellin, R848 and CpG. Cells were cultured for 72 hours with 5 μg/ml PGN, 10 μg/ml LPS, 50 ng/ml flagellin, 6 μg/ml R848, 10 μg/ml CpG ODNs, or with PBS as control. Cell recovery was assessed by crystal violet staining and expressed as a percent of control. None of those ligands significantly reduced cell recovery of any of the four breast cancer cell lines (Cama-1, BT483, SW527 and MCF-7). Although PGN had no effect on cell recovery, it induced secretion of IL-8 in certain cell lines, therefore establishing that the lack of cytotoxicity was not due to the absence of TLR triggering.

### Example 3

Cama-1 cells were analyzed for TLR3 mRNA expression in response to Poly IC. Cama-1 cells were cultured in complete medium (DMEM F12 containing 4.5 g/mL glucose and complemented with 2 mM L-glutamine, 10% fetal calf serum, 160 μg/mL gentalline, 2.5 mg/mL sodium bicarbonate) for 48 hours either alone or with LPS (5 μg/ml) and/or with Poly IC (5 μg/ml). The mRNA from each group of cells was extracted. The mRNA was then reverse-transcribed and PCR amplified for 35 cycles (as above in Example 1) with hTLR3 specific primers: TLR3F: aacgattcctttgcttggcttc (SEQ ID NO: 5) and TLR3R: gcttagatccagaatggtcaag (SEQ ID NO: 6). TLR3 mRNA could not be amplified from resting Cama-1 cells.

Amplified DNA from RT-PCR using hTLR3 specific primers was run on a gel. The gel showed TLR3 expression in the positive control (plasmid TLR3), in cells treated with Poly IC, and in cells treated with both Poly IC and LPS. The gel did not show TLR3 expression in cells treated with either LPS or with nothing (the negative control).

The data show that TLR3 mRNA expression is induced by Poly IC in human breast carcinoma Cama-1 cells. Therefore, Poly IC treatment upregulates the expression of its recognized receptor, TLR3, in certain tumor cell lines. On the other hand, treatment with LPS did not affect TLR3 mRNA expression in Cama-1 cells.

### Example 4

Two cell lines, the colon cancer cell line LS174T and the breast cancer cell line Cama-1, were analyzed for death and cell cycle changes. Cells were cultured for 48 hours in either the presence or absence of Poly IC (5 μg/ml). Following a 30-minute pulse with 1 μg/ml bromodeoxyuridine (BrdU), the cells were fixed overnight at 4° C. in 70% ethanol before staining with FITC-coupled anti-BrdU monoclonal antibody and 3 μg/ml propidium iodide. Cell death and the cell cycle were analyzed by flow cytometry (FACS). BrdU incorporation is a measure of proliferation, whereas propidium iodide staining allows the quantification of DNA content, in particular the subdiploid cell population undergoing apoptosis.

The data show that the percentage of LS174T cells that incorporated BrdU went from 27% before treatment to 9% after a 48 hour culture in the presence of Poly IC. Conversely, the percentage of LS174T cells that have a subdiploid DNA content went from 3% before treatment to 23% after a 48 hour culture in presence of Poly IC, indicative of a strong cytotoxicity of the Poly IC.

The data also show that the percentage of Cama-1 cells that incorporated BrdU went from 15% before treatment to 2% after a 48 hour culture in presence of Poly IC. Conversely, the percentage of Cama-1 cells that have a subdiploid DNA content went from 4% before treatment to 17% after a 48 hour culture in presence of poly IC, indicative of apoptosis triggered by the Poly IC.

These data indicate that upon treatment for 48 hours with Poly IC, both LS174T and Cama-1 cell lines stop dividing and undergo apoptosis.

### Example 5

To further investigate the effect of Poly IC treatment on breast tumor cell lines, cell death was analyzed in Cama-1 cells by annexin V staining. Cells were cultured for 24 hours either with or without 5 μg/ml of Poly IC. Apoptosis was measured by annexin V staining and flow cytometry. The data show that over 70% of the Cama-1 cells were stained by Annexin V, further demonstrating the apoptosis induced by Poly IC.

We also tried to determine the kinetics of Poly IC induced apoptosis. Cama-1 cells were cultured either with or without 5 μg/ml or 50 ng/ml of Poly IC. The percentage of apoptotic (annexin positive) cells in the culture were measured during the following 30 hours. The data show that untreated cells exhibited 15% of spontaneous apoptosis after 30 hours. However, 80% of the cells treated with Poly IC exhibited cell death. Specifically, Poly IC triggered apoptosis in Cama-1 cells beginning 9 hours after Poly IC addition and reaching up to 80% apoptotic cells after 30 hours of treatment.

We then tried to determine the effect that Poly IC has on human primary breast tumor cells. Freshly recovered tumor single cell suspensions were incubated with either PBS or Poly IC (50 μg/ml) for 48 hours. Apoptosis was measured by PI staining. The percentage represents the proportion of cells with low DNA content (subG0/G1 cells), i.e., apoptotic cells. The data show that 19.5% of the cells treated with PBS had a low DNA content whereas 38.6% of the cells treated with Poly IC had a low DNA content. Therefore, a similar cytotoxic effect of Poly IC was observed on human breast primary tumor cells.

### Example 6

TLR3 was analyzed for its role in Poly IC induced apoptosis. Cama-1 cells were transfected with siRNA corresponding to either: an irrelevant sequence (Scr RNA; sequence: ACUAGUUCACGAGUCACCUtt) (SEQ ID NO: 21), or hTLR3 (sequence: CAGUGUUGAACCUUACCCAtt) (SEQ ID NO: 22). siRNA transfections were performed for 5 hours in 1 mL OptiMEM™ medium containing 3 μg/mL lipofectamine 2000 and 100 nM siRNA. Cells were then washed in phosphate buffered saline solution (PBS) and cultured for 72 hours in complete medium before subsequent 48 hour treatment with 5 μg/mL Poly IC. The cell cycle was then analyzed by FACS after staining with ethidium bromide, as described in Example 3.

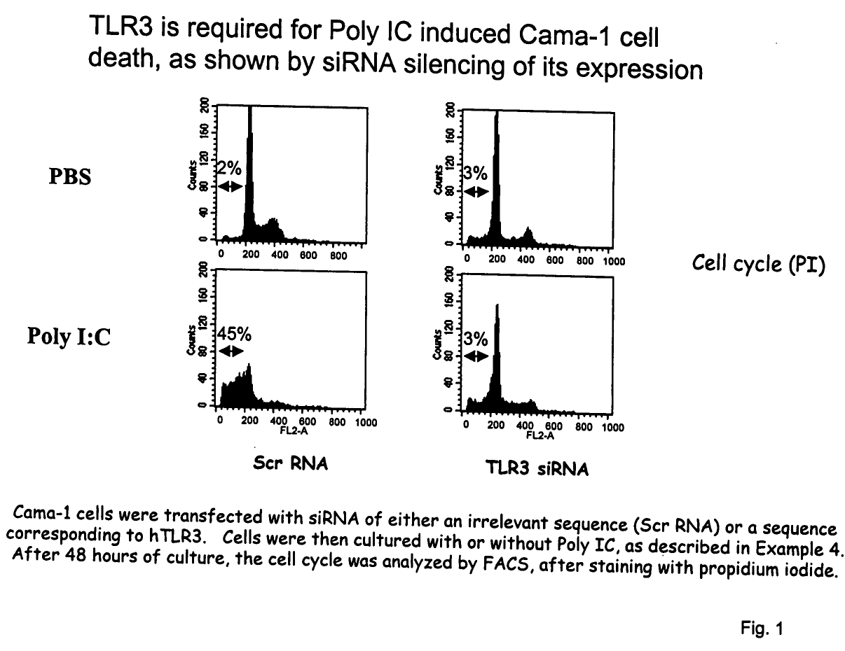

The results of these experiments are shown in FIG. 1. The data show that a 48 hour incubation of Cama-1 cells transfected with irrelevant, scrambled RNA in the presence of Poly,IC increased the percentage of subdiploid cells from 2% to 45%. However, a 48 hour incubation of Cama-1 cells transfected with hTLR3 siRNA in the presence of poly IC did not increase the percentage of subdiploid cells, which remained unchanged at 3%.

These data demonstrate that the apoptotic signal delivered to Cama-1 cells by Poly IC requires the expression of TLR3.

### Example 7

Poly AU was analyzed for its effects on apoptosis. Cama-1 cells were cultured for 48 hours either with PBS or with increasing concentrations of Poly AU ranging from 5 ng/ml to 50 μg/ml. Apoptosis was analyzed by measuring the percentage of annexin V positive cells. The data show that, similar to Poly IC, Poly AU triggers apoptosis.

### Example 8

We analyzed the effect of IFN on Poly IC induced apoptosis in vivo. TRP-Tag mice express SV40 T antigen in the retinal pigmented epithelium and typically develop eye tumors with complete penetrance within weeks from birth.

In these experiments, fourteen to sixteen TRP-Tag/IFNαβγR−/− mice per experiment (these are TPR-Tag mice that had been crossed to mice simultaneously deficient in the receptor for type I interferons (IFNαβR) and the receptor for type II interferon (IFNγR)) were treated on days 21, 23, 25, 27 and 29 by intravenous injections of either Poly IC (100 μg/dose) or PBS. The kinetics of visible eye tumor development was monitored 2-3 times per week.

The appearance of eye tumors was delayed by up to 21 days in mice treated with poly IC compared to mice treated with PBS. Since the mice used in these experiments had no functional interferon response system, the data show that Poly IC induced tumor growth inhibition is independent of type I and type II interferon in vivo.

### Example 9

In order to determine the pathway of Poly IC induced Cama-1 cell toxicity, RNA interference was used to efficiently downregulate expression of TRIF and PKR. Cama-1 cells were plated in 6 well plates at 3×10⁵ cells per well. After overnight adherence, siRNA transfections were performed for 5 hours in OptiMEM medium (Life technologies) containing 3 μg/mL lipofectamine 2000 (Invivogen) and 100 nM siRNA. Cells were transfected with either MOCK (water), control scrambled duplex (scr) siRNA, TRIF siRNA or PKR siRNA.

siRNA duplexes specific for PKR was purchased from Dharmacon (Lafayette, Colo.) as SMART-Pools. TRIF siRNA was purchased from the same supplier as single oligoduplexes 5′-GCUCUUGUAUCUGMGCAC-3′ (SEQ ID NO: 23). TLR3 and TRIF expression was assessed by PCR (35 cycles: 1 min. 94° C., 1 min. 55° C., 2 min. 72° C.) with Taq PCR ReadyMix (Sigma-Aldrich) using the following primers: 5′-AACGATTCCTTTGCTTGGCTTC-3′ (SEQ ID NO: 24) (forward)/5′-GCTTAGATCCAGAATGGTCMG-3′ (SEQ ID NO: 25) (reverse) for TLR3 and 5′-ACTTCCTAGCGCCTTCGACA-3′ (SEQ ID NO: 26) (forward)/5′-ATCTTCTACAGAAAGTTGGA-3′ (SEQ ID NO: 27) (reverse) for TRIF. Expression of PKR was assessed by Western Blot. For TRIF mRNA, PCR was performed after another 24 hour culture either with or without 5 μg/ml of Poly IC.

The data show that RNA interference was used to efficiently downregulate expression of TRIF and PKR.

72 hours after siRNA transfection, Cama-1 cells were cultured for another 24 hours either with or without 5 μg/ml Poly IC. Apoptosis was measured by annexin V staining and expressed as a percentage of apoptotic cells in culture. On average, 10% of control cells (MOCK and scr) that were untreated underwent apoptosis. In contrast, about 75% of control cells (MOCK and scr) that were treated with Poly IC underwent apoptosis. In the TRIF siRNA groups, untreated cells exhibited 10% apoptotic cells, whereas cells treated with TRIF siRNA exhibited 20% apoptotic cells. Finally, in the PKR siRNA group, untreated cells exhibited 10% apoptotic cells, whereas cells treated with PKR siRNA exhbited 80% apoptotic cells.

Therefore, treatment with siRNA to TRIF virtually abrogated Poly IC induced apoptosis, whereas cell death occurred normally in the absence of PKR expression.

These data clearly demonstrate that Poly IC induced apoptosis in Cama-1 cells is both mediated by both TLR3 and TRIF, and is PKR independent.

### Example 10

To further investigate TLR3 mediated cytotoxicity, the involvement of the signaling molecules IRAK4 and TRAF6, both downstream mediators of TLR signaling, were assessed. Cama-1 cells were plated in 6 well plates at 3×10⁵ cells per well. After overnight adherence, siRNA transfections were performed for 5 hours in OptiMEM medium (Life technologies) containing 3 μg/mL lipofectamine 2000 (Invivogen) and 100 nM siRNA. Cells were transfected with either control scrambled duplex (scr) siRNA, IRAK-4 siRNA or TRAF-6 siRNA. Cells were then washed and cultured for 72 hours in complete medium before treatment with Poly IC and apoptosis analysis. siRNA duplexes specific for IRAK4 and TRAF6 were purchased from Dharmacon (Lafayette, Colo.) as SMART-Pools.

Expression of IRAK-4 and TRAF6 was analyzed by Western Blot. The Western Blot shows that IRAK-4 and TRAF6 siRNA abolishes the expression of the corresponding proteins.

72 hours after siRNA transfection, Cama-1 cells were cultured for another 24 hours either with or without 5 μg/ml Poly IC. Apoptosis was measured by annexin V staining and expressed as a percentage of apoptotic cells in culture. On average, 10% of control cells (scr) that were untreated underwent apoptosis. In contrast, about 75% of control cells (scr) that were treated with Poly IC underwent apoptosis. In the IRAK-4 siRNA groups, cultures exhibited only 20% apoptotic cells, whereas in the TRAF6 siRNA groups, 75% of the cells were apoptitic at the end of the culture. In the TRAF6 siRNA groups, untreated cells exhibited 15% apoptotic cells, whereas cells treated with TRAF6 siRNA exhibited 75% apoptotic cells.

The data show that inhibition of IRAK-4 expression resulted in inhibited TLR3-mediated cellular toxicity. However, inhibition of TRAF6 expression did not result in inhibited TLR3-mediated cellular toxicity. This finding was unexpected because TRAF6 is thought to be located downstream of IRAK-4 in the TLR signaling pathway. Therefore, this suggests that TLR3 could signal via IRAK-4 to activate a TRAF6 independent apoptotic pathway.

In parallel, IL-6 concentration in the supernatants of siRNA transfected Cama-1 cells cultured for 24 hours either with or without 5 μg/ml of Poly IC was determined by ELISA. The data show that for the scr group, untreated and treated cells had IL-6 concentrations (pg/ml/10⁶ cells) of 10 and 110, respectively. In the siRNA IRAK-4 group, untreated and treated cells had IL-6 concentrations (pg/ml/10⁶ cells) of 10 and 40, respectively. In the siRNA TRAF6 group, untreated and treated cells had IL-6 concentrations (pg/ml/10⁶ cells) of 10 and 20, respectively. These data show that both IRAK-4 and TRAF6 were required for cytokine production.

### Example 11

The involvement of type 1 interferon in TLR3 mediated apoptosis was evaluated. Cama-1 cells were incubated with 5 μg/ml Poly IC for either 0 hours, 1 hour, 6 hours, 18 hours or 24 hours. The presence of IFN-β, phosphorylated Stat1 (tyrosine 701) (P-Stat-1) and total Stat-1 in the cell lysate were analyzed by Western Blot.

The data show that IFN-fi production was strongly induced upon Poly IC treatment. Also, Stat1 phosphorylation was observed. These observations demonstrate that type I IFN signaling was triggered by Poly IC in Cama-1 cells. Interestingly, Stat1 phosphorylation was at a maximum after 6 hours of Poly IC treatment, when IFN-β production was still hardly detectable.

In another experiment, Cama-1 cells were pre-incubated for 1 hour with 20 μg/ml of either neutralizing IFN type I receptor mAb (anti-IFN R1) or isotype control (mouse IgG1). The cells were then cultured for 24 hours either with or without 5 μg/ml Poly IC or with a mixture of 1000 U/ml each of IFN-α or IFN-β. Apoptosis was measured by annexin V staining and expressed as a percentage of apoptotic cell in the culture.

In the absence of antibody, the untreated, Poly IC and IFNα/β, treated cells exhibited 10%, 70% and 20% apoptotic cells, respectively. In the migG1 group, the untreated, Poly IC and IFNα/β treated cells exhibited 10%, 70% and 20% apoptotic cells, respectively. In the anti-IFN R1 group, the untreated, Poly IC and IFNα/β treated cells exhibited 10%, 30% and 15% apoptotic cells, respectively.

The data show that neutralization of type I IFN receptors with a specific monoclonal antibody significantly reduced Poly IC induced apoptosis. This demonstrates that type I IFNs are necessary for TLR3 mediated apoptosis.

Treatment of Cama-1 cells with a mixture of IFNα and IFNβ was not able to induce significant apoptosis. This shows that type I IFN signaling was needed for TLR3 triggered cytotoxicity, but is not sufficient to induce cell death alone.

### Example 12

We tried to determine whether TNF-α plays a role in TLR3 mediated apoptosis. Cama-1 cells were pre-incubated either with or without 20 μg/ml of neutralizing anti TNF-α mAb or 10 μg/ml CHX. The cells where then cultured either with or without 5 μg/mi Poly IC or 25 ng/ml of TNF-α. Apoptosis was measured by annexin V staining and expressed as a percentage of apoptotic cells in culture.

In the absence of antibody, the untreated, Poly IC and TNF-α treated cells exhibited 10%, 70% and 40% apoptotic cells, respectively. In the anti-TNF-α mAb group, the untreated, Poly IC and TNF-α treated cells exhibited 10%, 65% and 10% apoptotic cells, respectively. In the CHX group, the untreated, Poly IC and TNF-α treated cells exhibited 15%, 40% and 70% apoptotic cells, respectively.

The data show that a neutralizing anti-TNF-α antibody, which protected Cama-1 cells from TNF-α induced apoptosis, had no effect on Poly IC triggered cell death. Therefore, TNF-α does not play a role in TLR3 mediated apoptosis.

As stated above, Cama-1 cells were pre-treated with the general transcriptional inhibitor CHX, which is known to sensitize cells to TNF-α induced apoptosis by blocking the NFκB controlled survival program.

The data show that CHX significantly sensitized Cama-1 cells to TNF-α induced apoptosis. In contrast, CHX partially protected the cells against Poly IC induced apoptosis. This confirms that different mechanisms were triggered by these two pro-apoptotic stimuli.

RNA interference was then used to assess the involvement of NFκB in TLR3 mediated apoptosis. Cama-1 cells transfected 72 hours earlier with siRNA to p65 or scrambled control duplex (scr) were cultured for 24 hours either with or without 50 ng/ml or 5 μg/ml of Poly IC. Extinction of p65 protein expresion before Poly IC treatment was assessed by Western Blot. Apoptosis was measured by annexin V staining. Results were expressed as a percent of apoptotic cells in culture.

In the scr group, the untreated, Poly IC (50 ng/ml) and Poly IC (5 μg/ml) treated cells exhibited 10%, 20% and 70% apoptotic cells, respectively. In the siRNA p65 group, the untreated, Poly IC (50 ng/ml) and Poly IC (5 μg/ml) treated cells exhibited 10%, 10% and 20% apoptotic cells, respectively.

The data show that inhbition of NFKB p65 expression by siRNA led to a significant protection against Poly IC induced cellular toxicity. This confirms the pro-apoptotic role of NFκB in Poly IC triggered apoptosis.

Collectively, these results demonstrate that TNF-α secretion is not responsible for Poly IC induced apoptosis. In addition, these results demonstrate a pro-apoptotic role of NFκB in TLR3 mediated apoptosis, which contrasts with its anti-apoptotic effect upon TNFtreatment.

### Example 13

We next addressed the role of caspases in apoptosis. Cama-1 cells were pre-incubated with 25 μM of the general caspase inhibitor z-VAD-fmk or DMSO for 1 hour before culture for 24 hours with or without 5 μg/ml Poly IC or 25 ng/ml TNF-α (used as a positive control). Apoptosis was measured by annexin V staining and expressed as a percentage of apoptotic cell in the culture.

In the DMSO group, the untreated, Poly IC and TNF-α treated cells exhibited 10%, 70% and 40% apoptotic cells, respectively. In the z-VAD-fmk group, the untreated, Poly IC and TNF-α treated cells exhibited 10%, 30% and 10% apoptotic cells, respectively.

The data show that inhibition of caspase activity by the broad caspase inhibitor z-VAD-fmk greatly reduced Poly IC induced apoptosis. This suggests a major role for caspases in TLR3 triggered cytotoxicity.

In another experiment, lysates from cells obtained above were analyzed by Western Blot for cleavage of PARP, Caspase 3 and Caspase 8.

The data show that cleavage of PARP, a hallmark of caspase-dependent apoptosis, occurred in Cama-1 cells upon Poly IC treatment. This confirms the involvement of caspases in TLR3 mediated apoptosis. Indeed, caspase 3 was activated upon Poly IC treatment, as evidenced by Western Blot analysis.

### Example 14

We tried to further investigate whether any synergy exists between TLR3 ligands and type I IFN. The primary breast carcinoma cells SKBr3 were plated in 6 well plates at 3×10⁵ cells per well. After overnight adherence, siRNA transfections were performed for 5 hours in OptiMEM medium (Life technologies) containing 3μg/mL lipofectamine 2000 (Invivogen) and 100 nM siRNA. Cells were transfected with either MOCK (water), TLR3 siRNA or PKR siRNA. Cells were then washed and cultured for 72 hours in complete medium before 24 hour treatment with 50 μg/ml Poly IC and apoptosis analysis.

In the MOCK group, the untreated and Poly IC (50 μg/ml) treated cells exhibited 10% and 22% apoptotic cells, respectively. In the TLR3 siRNA group, the untreated and Poly IC (50 μg/ml) treated cells exhibited 8% and 13% apoptotic cells, respectively. In the PKR siRNA group, the untreated and Poly IC (50 μg/ml) treated cells exhibited 12% and 22% apoptotic cells, respectively.

The data show that the breast adenocarcinoma cell line SKBr3 underwent partial apoptosis when treated with Poly IC. In addition, the data show that pre-treatment of the cells with TLR3 siRNA abolished apoptosis, while the PKR siRNA did not have a protective effect.

In another experiment, we tried to determine whether IFN and Poly IC acted synergistically to induce apoptosis. SKBr3 cells were untreated or pre-treated with either 10 U/ml or 100 U/ml of a low dose mixture of IFN-α or IFN-β. Poly IC was administered in the following doses: 0, 0.5, 5 and 50 μg/ml for 48 hours.

The data show that in the untreated Poly IC group, the untreated, IFN-α/β (10 U/ml) and IFN-α/β (100 U/ml) treated cells exhibited 10%, 14% and 22% apoptotic cells, respectively. In the 0.5 μg/ml Poly IC group, the untreated, IFN-α/β (10 U/ml) and IFN-α/β (100 U/ml) treated cells exhibited 15%, 45% and 55% apoptotic cells, respectively. In the 5 μg/ml Poly IC group, the untreated, IFN-α/β (10 U/ml) and IFN-α/β (100 U/ml) treated cells exhibited 20%, 55% and 60% apoptotic cells, respectively. In the 50 μg/ml Poly IC group, the untreated, IFN-α/β (10 U/mI) and IFN-α/β (100 U/ml) treated cells exhibited 20%, 55% and 60% apoptotic cells, respectively.

Therefore, IFN was able to act synergistically with Poly IC to induce apoptosis. This synergy had two manifestations: 1) when pre-treated, SKBr3 cells became sensitive to Poly IC induced apoptosis at concentrations that were one hundred fold lower than non-pretreated cells; and 2) the percentage of SKBr3 cells that were induced to apoptosis by Poly IC increased from 22% to 66% after type I IFN pre-treatment.

In conclusion, type I IFN pre-treatment sensitizes SKBr3 breast adenocarcinoma cells to TLR3 mediated Poly IC induced apoptosis. Therefore, pre-treatment of breast cancer patients with low dose type I IFN not only increases the efficacy of Poly IC treatment, but also allows the recruitment of patients that wouldn't otherwise have the benefit from Poly IC. Patients could also be treated before surgery with low dose type I IFN to increase the percentage of tumors that will be scored positive by immuno-histology on biopsies, and that will become responsive to TLR3 ligands. In addition, the combination of low dose type I IFN and low dose Poly IC may be more effective than a higher dose of Poly IC alone. This combination may also reduce the risk of side effects.

We claim:

1) A method for treating cancer comprising:
a) selecting a patient that has a TLR expressing cancer, and
b) administering to said patient a therapeutically effective amount of a TLR ligand.

2) The method of claim 1 wherein said ligand is an agonist or an antagonist.

3) A method for inducing apoptosis of a tumor cell comprising:
a) selecting a TLR expressing tumor cell, and
b) contacting said cell with a TLR ligand in an amount effective to induce apoptosis in said cell.

4) The method of claim 3 wherein said ligand is an agonist or an antagonist.

5) A method for treating cancer comprising:
a) selecting a patient that has a TLR3 expressing cancer; and
b) administering to said patient a therapeutically effective amount of a TLR3 ligand.

6) The method of claim 5 wherein said ligand is an agonist or an antagonist.

7) The method of claim 6 wherein said agonist is Poly IC.

8) The method of claim 6 wherein said agonist is Poly AU.

9) The method of claim 6 wherein said antagonist is an antibody or fragment thereof.

10) The method of claim 5 wherein said TLR3 expressing cancer is breast cancer.

11) The method of claim 5 wherein said TLR3 expressing cancer is colon cancer.

12) The method of claim 5, wherein said method further comprises administering to said patient a chemotherapeutic agent or a cancer treatment.

13) The method of claim 5, wherein said method further comprises administering to said patient a low dose of type I IFN prior to administration of TLR3 ligand.

14) A method for inducing apoptosis of a tumor cell comprising:
a) selecting a TLR3 expressing tumor cell, and
b) contacting said cell with a TLR3 ligand in an amount effective to induce apoptosis in said cell.

15) The method of claim 14 wherein said ligand is an agonist or an antagonist.

16) The method of claim 15 wherein said agonist is Poly IC.

17) The method of claim 15 wherein said agonist is Poly AU.

18) The method of claim 15 wherein said antagonist is an antibody or fragment thereof.

19) The method of claim 14 wherein said TLR3 expressing tumor cell is a breast cancer cell.

20) The method of claim 14 wherein said TLR3 expressing tumor cell is a colon cancer cell.

21) The method of claim 14, wherein said method further comprises contacting said cell with a chemotherapeutic agent or a cancer treatment.

22) The method of claim 14, wherein said method further comprises contacting said cell with a low dose of type I IFN prior to administration of TLR3 ligand.


SEQUENCE LISTING (27 sequences)

SEQ ID NO: 1 - DNA, 23 bp, Homo sapiens
caggatcaaggtacttgatcttc

SEQ ID NO: 2 - DNA, 23 bp, Homo sapiens
tttctctcatgaaggcaaatctg

SEQ ID NO: 3 - DNA, 20 bp, Homo sapiens
ctcaggagcagcaagcactg

SEQ ID NO: 4 - DNA, 21 bp, Homo sapiens
atcttccgcagcttgcagaag

SEQ ID NO: 5 - DNA, 22 bp, Homo sapiens
aacgattcctttgcttggcttc

SEQ ID NO: 6 - DNA, 22 bp, Homo sapiens
gcttagatccagaatggtcaag

SEQ ID NO: 7 - DNA, 22 bp, Homo sapiens
ctcagaatgactttgcttgtac

SEQ ID NO: 8 - DNA, 22 bp, Homo sapiens
gcaggacaatgaagatgatacc

SEQ ID NO: 9 - DNA, 21 bp, Homo sapiens
cgaacctcatccacttatcag

SEQ ID NO: 10 - DNA, 23 bp, Homo sapiens
gtgaactttagggactttaagac

SEQ ID NO: 11 - DNA, 21 bp, Homo sapiens
ccaatgtacctgtgagctaag

SEQ ID NO: 12 - DNA, 22 bp, Homo sapiens
ccactcactctggacaaagttg

SEQ ID NO: 13 - DNA, 23 bp, Homo sapiens
ggatctgtctttcaattttgaac

SEQ ID NO: 14 - DNA, 20 bp, Homo sapiens
ccaaggtctgcccatacttg

SEQ ID NO: 15 - DNA, 23 bp, Homo sapiens
gctatccttgtgatgagaaaaag

SEQ ID NO: 16 - DNA, 21 bp, Homo sapiens
gcattgaagcacctcggacag

SEQ ID NO: 17 - DNA, 20 bp, Homo sapiens
actgtttcgccctctcgctg

SEQ ID NO: 18 - DNA, 21 bp, Homo sapiens
gccagcacaaacagcgtcttg

SEQ ID NO: 19 - DNA, 21 bp, Homo sapiens
ttgttcagagctgccaggaag

SEQ ID NO: 20 - DNA, 24 bp, Homo sapiens
gcaaagtagaattcataatggcac

SEQ ID NO: 21 - DNA, 21 bp, Artificial
  siRNA corresponding to an irrelevant sequence
acuaguucacgagucaccutt

SEQ ID NO: 22 - DNA, 21 bp, Homo sapiens
caguguugaaccuuacccatt

SEQ ID NO: 23 - RNA, 19 nt, Artificial
  TRIF siRNA
gcucuuguaucugaagcac

SEQ ID NO: 24 - DNA, 22 bp, Artificial
  TLR3 forward primer
aacgattcctttgcttggcttc

SEQ ID NO: 25 - DNA, 22 bp, Artificial
  TLR3 reverse primer
gcttagatccagaatggtcaag

SEQ ID NO: 26 - DNA, 20 bp, Artificial
  TRIF forward primer
acttcctagcgccttcgaca

SEQ ID NO: 27 - DNA, 20 bp, Artificial
  TRIF reverse primer
atcttctacagaaagttgga